(19) United States
(12) Patent Application Publication
(10) Pub. No.: US 2022/0203073 A1
(43) Pub. Date: Jun. 30, 2022

(54) CATHETER ASSEMBLY

(71) Applicant: TERUMO KABUSHIKI KAISHA, Tokyo (JP)

(72) Inventor: Shinichi MIZUNO, Nakakoma-gun (JP)

(73) Assignee: TERUMO KABUSHIKI KAISHA, Tokyo (JP)

(21) Appl. No.: 17/696,210

(22) Filed: Mar. 16, 2022

Related U.S. Application Data
(63) Continuation of application No. PCT/JP2020/033713, filed on Sep. 7, 2020.

(30) Foreign Application Priority Data
Sep. 25, 2019 (JP) 2019-173619

(51) Int. Cl. A61M 25/06; A61M 25/00

(52) U.S. Cl. CPC A61M 25/0606; A61M 25/0097; A61M 25/065

(57) ABSTRACT

A catheter assembly includes: an inner needle including, at a distal end portion of the inner needle, a blade surface that is inclined with respect to an axis of the inner needle; a needle hub fixed to a proximal end portion of the inner needle; a tubular catheter shaft through which the inner needle is inserted; a hollow catheter hub through which the inner needle is inserted and that is fixed to a proximal end portion of the catheter shaft; and a restriction mechanism configured to restrict relative axial movement of the catheter hub and the needle hub along an axial direction in an initial state of the catheter assembly. The restriction mechanism is configured such that the restriction of the relative axial movement is released when the needle hub is rotated with respect to the catheter hub along a circumferential direction of the inner needle from the initial state.

### CROSS-REFERENCE TO RELATED APPLICATIONS

The present application is a bypass continuation of PCT Application No. PCT/JP2020/033713, filed on Sep. 7, 2020, which claims priority to Japanese Application No. 2019-173619, filed on Sep. 25, 2019. The contents of these applications are hereby incorporated by reference in their entireties.

### BACKGROUND

The present disclosure relates to a catheter assembly.

JP 2009-233007 A discloses a catheter assembly (indwelling needle) including a needle member and a catheter member. The needle member includes an inner needle and a hollow needle hub fixed to a proximal end portion of the inner needle. The catheter member includes a tubular catheter shaft through which the inner needle is inserted, and a hollow catheter hub through which the inner needle is inserted and that is fixed to a proximal end portion of the catheter shaft. A blade surface inclined with respect to the axis of the inner needle is formed at the distal end portion of the inner needle.

### SUMMARY

In a procedure using the catheter assembly, after puncturing the blood vessel with the inner needle and the catheter shaft in a state in which the inner needle protrudes from the distal end of the catheter shaft (the state in which the blade surface of the inner needle is directed upward), the inner needle is removed from the catheter shaft while the catheter shaft is indwelled in the blood vessel.

However, if the catheter assembly is moved back and forth to probe the blood vessel at the time of the puncture operation, the inner needle and the catheter shaft may move relatively axially along the axial direction, and the blade edge positioned at the most distal end of the inner needle may pierce the catheter shaft.

In addition, because the position of the blade edge is located at a lower side in a state in which the puncture operation is completed, if the inner needle is advanced with respect to the catheter shaft during the removal operation, the blade edge may pierce the catheter shaft. In particular, when the catheter shaft is curved downward at the time of the removal operation, the blade edge may easily pierce the catheter shaft by the advancing operation of the inner needle.

Embodiments of the present invention have been developed in view of such problems, and an object of certain embodiments of the present invention is to provide a catheter assembly capable of suppressing a blade edge from piercing a catheter shaft in a puncturing operation and a removal operation.

According to one aspect of the present invention, a catheter assembly includes an inner needle, a needle hub fixed to a proximal end portion of the inner needle, a tubular catheter shaft through which the inner needle is inserted, and a hollow catheter hub through which the inner needle is inserted and that is fixed to a proximal end portion of the catheter shaft, a blade surface inclined with respect to an axis of the inner needle being formed at a distal end portion of the inner needle, the catheter assembly further including a restriction mechanism that restricts relative axial movement of the catheter hub and the needle hub along an axial direction in an initial state of the catheter assembly, wherein the restriction mechanism is formed so that the restriction of the relative axial movement is released when the needle hub is rotated with respect to the catheter hub along a circumferential direction of the inner needle from the initial state of the catheter assembly.

According to the present invention, because the relative axial movement of the catheter hub and the needle hub is restricted by the restriction mechanism in the initial state of the catheter assembly, the relative axial movement of the inner needle and the catheter hub is restricted in the puncture operation. Therefore, in the puncturing operation, it is possible to inhibit the blade edge from piercing the catheter shaft.

In addition, after completion of the puncture operation, the user rotates the needle hub with respect to the catheter hub along the circumferential direction of the inner needle to release the restriction of the relative axial movement. As a result, because the inner needle rotates around the axis, the direction of the blade surface is changed. That is, the position of the blade edge moves upward. Therefore, even when the catheter shaft is curved downward during the removal operation, the blade edge can be separated from the inner surface of the catheter shaft. Therefore, in the removal operation, it is possible to inhibit the blade edge from piercing the catheter shaft.

### BRIEF DESCRIPTION OF THE DRAWINGS

FIG. 1 is a perspective view of a catheter assembly according to an embodiment of the present invention.

FIG. 2 is an exploded perspective view of the catheter assembly of FIG. 1.

FIG. 3 is a longitudinal sectional view of the catheter assembly of FIG. 1.

FIG. 4 is a partially enlarged view of the catheter assembly of FIG. 3.

FIG. 5 is a transverse cross-sectional view taken along line V-V of FIG. 4.

FIG. 6 is a perspective view from a proximal end side of the catheter hub of FIG. 1.

FIG. 7 is a partially enlarged perspective view of the needle hub of FIG. 1.

FIG. 8 is an explanatory view of a puncturing operation of the catheter assembly of FIG. 1.

FIG. 9 is a first explanatory view of a removal operation of the catheter assembly of FIG. 1.

FIG. 10 is a second explanatory view of a removal operation of the catheter assembly of FIG. 1.

FIG. 11 is a third explanatory view of a removal operation of the catheter assembly of FIG. 1.

### DETAILED DESCRIPTION

Hereinafter, preferred embodiments of a catheter assembly according to the present invention will be described with reference to the accompanying drawings.

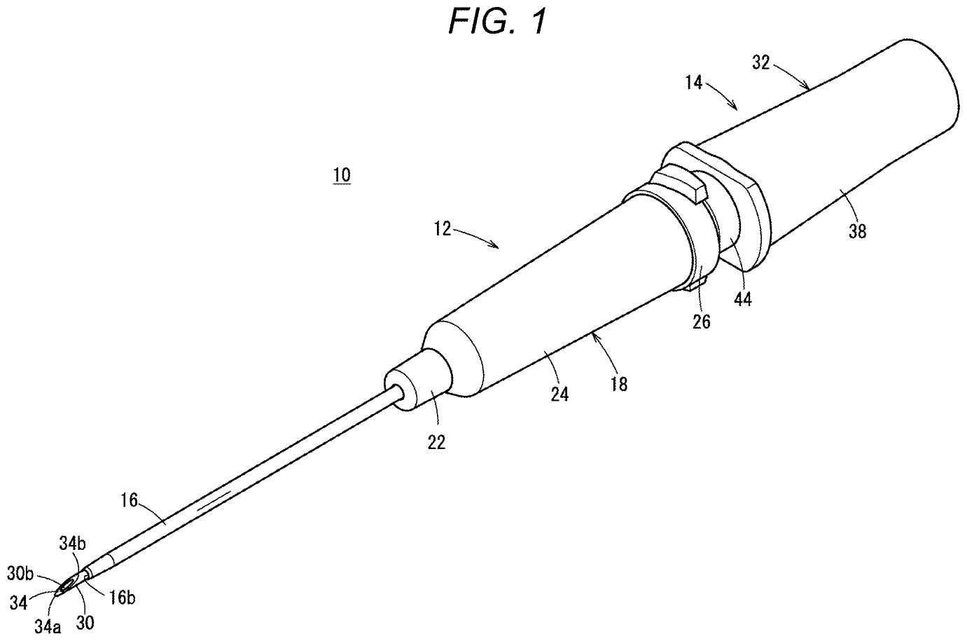

A catheter assembly 10 according to an embodiment of the present invention is configured as an indwelling needle for administering an infusion solution (medicinal solution) into a blood vessel of a patient (living body). As illustrated in FIGS. 1 to 3, the catheter assembly 10 includes a catheter member 12 and a needle member 14. The catheter member 12 includes a catheter shaft 16, and a catheter hub 18 fixed to a proximal end portion of the catheter shaft 16.

The catheter shaft 16 is a tubular member having flexibility and configured to be continuously inserted into a blood vessel of a patient. The catheter shaft 16 has a lumen 16a extending along the axial direction over the entire length thereof. A distal end opening 16b communicating with the lumen 16a is formed at the distal end of the catheter shaft 16.

A constituent material of the catheter shaft 16 is not particularly limited, but a resin material having transparency, particularly a soft resin material is suitable. Examples thereof include a fluorine-based resin such as polytetrafluoroethylene (PTFE), ethylene-tetrafluoroethylene copolymer (ETFE), and perfluoroalkoxy fluorine resin (PFA), an olefin-based resin such as polyethylene and polypropylene or a mixture thereof, polyurethane, polyester, polyamide, polyether nylon resin, a mixture of an olefin-based resin and an ethylene-vinyl acetate copolymer, and the like.

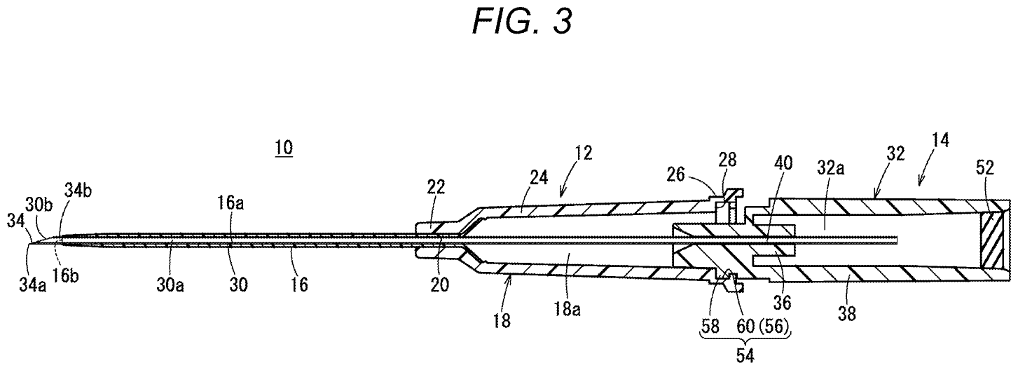

The catheter hub 18 is formed in a hollow shape (cylindrical shape). As illustrated in FIG. 3, a first attachment hole 20 to which a proximal end portion of the catheter shaft 16 is attached is formed at a distal end portion of the catheter hub 18. The outer peripheral surface of the proximal end portion of the catheter shaft 16 is fixed to the wall surface forming the first attachment hole 20 by an appropriate fixing means such as caulking, fusion, or adhesion.

A lumen 18a communicating with the lumen 16a of the catheter shaft 16 is formed on the proximal end side of the catheter hub 18 with respect to the first attachment hole 20. The lumen 18a of the catheter hub 18 is open at the proximal end of the catheter hub 18. Although not illustrated, a hemostasis valve, a seal member, and a plug may be disposed in the lumen 18a of the catheter hub 18. The catheter hub 18 has transparency such that blood flowing into the lumen 18a of the catheter hub 18 can be visually recognized from the outside of the catheter hub 18. That is, the catheter hub 18 is made of a transparent or translucent material.

The catheter hub 18 is preferably made of a material harder than the catheter shaft 16. A constituent material of the catheter hub 18 is not particularly limited, but for example, thermoplastic resins such as polypropylene, polycarbonate, polyamide, polysulfone, polyarylate, a methacrylate-butylene-styrene copolymer, polyurethane, an acrylic resin, and an ABS resin can be suitably used.

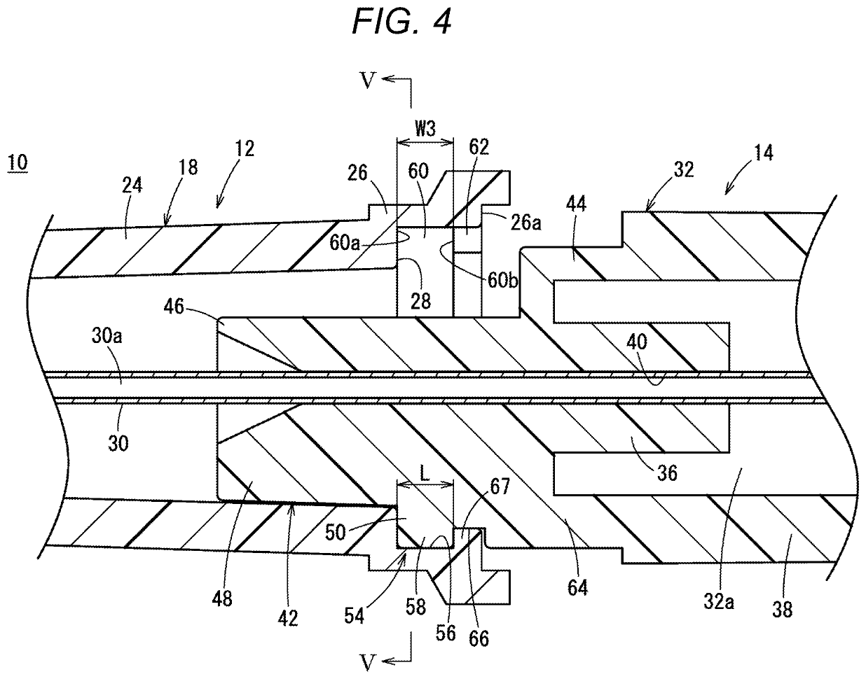

As illustrated in FIGS. 1 to 3, the catheter hub 18 includes a distal cylindrical portion 22 in which the first attachment hole 20 is formed, an intermediate cylindrical portion 24 provided at a proximal end portion of the distal cylindrical portion 22, and a proximal cylindrical portion 26 provided at a proximal end portion of the intermediate cylindrical portion 24. The outer diameter of the distal cylindrical portion 22 is smaller than the outer diameter of the intermediate cylindrical portion 24. The outer diameter of the proximal cylindrical portion 26 is larger than the outer diameter of the intermediate cylindrical portion 24. In FIG. 4, the inner surface of the proximal cylindrical portion 26 is larger in diameter than the inner surface of the intermediate cylindrical portion 24. That is, a stepped surface 28 extending in an annular shape is formed at the proximal end of the intermediate cylindrical portion 24.

In FIGS. 1 to 3, the needle member 14 includes an inner needle 30 and a needle hub 32 fixed to a proximal end portion of the inner needle 30. The inner needle 30 is a tubular member having rigidity and being capable of puncturing the skin of the patient. As illustrated in FIG. 3, the inner needle 30 has a lumen 30a extending along the axial direction. The inner needle 30 is inserted into the lumen 16a of the catheter shaft 16 and the lumen 18a of the catheter hub 18 in the initial state (assembled state) of the catheter assembly 10.

Examples of the constituent material of the inner needle 30 include metal materials such as stainless steel, aluminum, an aluminum alloy, titanium, and a titanium alloy. The inner needle 30 is formed sufficiently longer than the catheter shaft 16 and protrudes from the distal end opening 16b of the catheter shaft 16 in the initial state of the catheter assembly 10 (see FIGS. 1 and 3).

In FIG. 1, a blade surface 34 inclined with respect to the axis of the inner needle 30 is formed at the distal end portion of the inner needle 30. The blade surface 34 has a blade edge 34a located at the most distal end of the inner needle 30 and a blade base 34b located at the proximal end of the blade surface 34 in the axial direction. The blade surface 34 extends in a flat shape. However, the blade surface 34 may extend to be curved in a concave shape. An elliptical distal end opening 30b communicating with the lumen 30a of the inner needle 30 is formed on the blade surface 34. The distal end opening 30b of the inner needle 30 extends along the extending direction of the blade surface 34. A blood introduction groove (not illustrated) for guiding blood to the lumen 18a of the catheter hub 18 through between the inner needle 30 and the catheter shaft 16 may be formed on the outer surface of the inner needle 30.

In FIG. 3, the needle hub 32 is formed in a hollow shape (cylindrical shape). The constituent material of the needle hub 32 may be the same as the constituent material of the catheter hub 18 described above. The needle hub 32 includes an inner needle support portion 36 forming a distal end portion thereof and a needle hub body 38 extending from the inner needle support portion 36 to the proximal end side.

A second attachment hole 40 to which the proximal end portion of the inner needle 30 is attached is formed in the inner needle support portion 36. The outer peripheral surface of the proximal end portion of the inner needle 30 is fixed to the wall surface forming the second attachment hole 40 by an appropriate fixing means such as fusion, adhesion, or fitting.

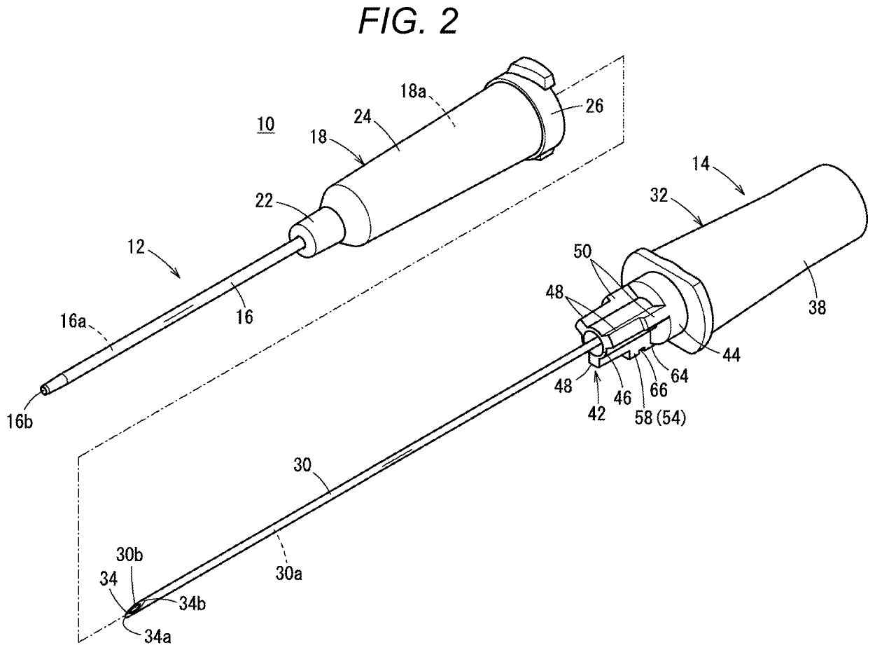

In FIGS. 2, 4, and 7, the distal end portion of the inner needle support portion 36 is provided with an insertion portion 42 to be inserted into the lumen 18a of the catheter hub 18 in the initial state of the catheter assembly 10, and a connecting portion 44 connecting the insertion portion 42 and the needle hub body 38. The insertion portion 42 includes an annular portion 46 forming a part of the inner needle support portion 36, three first protruding portions 48 protruding radially outward from an outer surface of the annular portion 46, and three second protruding portions 50 protruding radially outward from the protruding end surfaces of the first protruding portions 48.

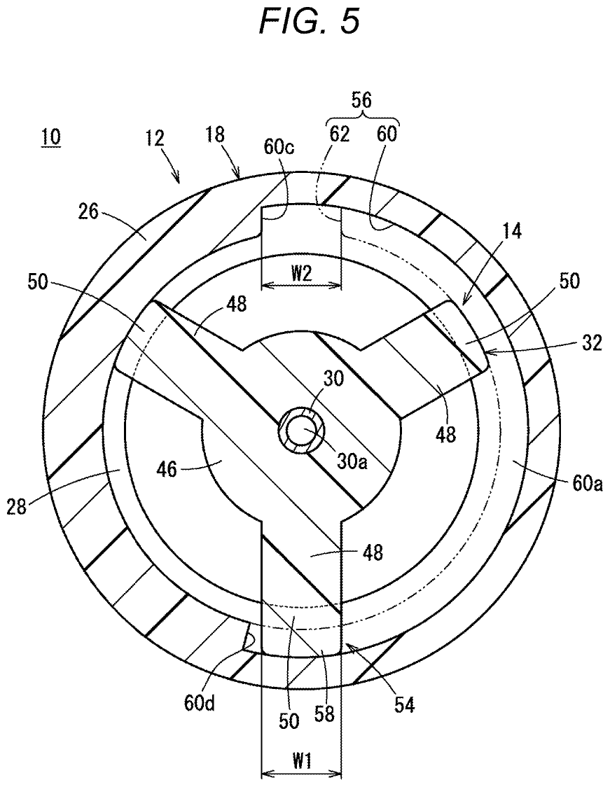

The three first protruding portions 48 are provided at equal intervals (120° intervals) in the circumferential direction of the annular portion 46 (see FIG. 5). Each of the first protruding portions 48 extends over the entire length of the annular portion 46 along the axial direction of the needle hub 32. That is, each of the first protruding portions 48 extends from the most distal end of the insertion portion 42 to the distal end surface of the connecting portion 44. The second protruding portion 50 is provided on the proximal end side of the first protruding portion 48. In other words, the proximal end of the second protruding portion 50 is connected to the distal end surface of the connecting portion 44. The width dimension of the second protruding portion 50 (dimension along the circumferential direction of the annular portion 46) is the same as the width dimension of the first protruding portion 48.

In FIG. 4, a portion of each of the first protruding portions 48 on the distal side of the second protruding portion 50 is located in the intermediate cylindrical portion 24 together with the annular portion 46 in the initial state of the catheter assembly 10. In the initial state of the catheter assembly 10, each of the second protruding portions 50 faces (is in contact with or in proximity to) the stepped surface 28 of the catheter hub 18 and is located in the proximal cylindrical portion 26. The number, position, shape, and size of the first protruding portions 48 and the second protruding portions 50 can be appropriately changed.

As illustrated in FIGS. 1 to 3, the needle hub body 38 is formed in a size and shape that can be easily gripped by the user. In the needle hub 32, a lumen 32a that communicates with the lumen 30a of the inner needle 30 and into which blood guided from the inner needle 30 flows is formed (see FIG. 3). The lumen 32a of the needle hub 32 is provided with a filter member 52 that permits the flow of air and blocks the flow of blood. At least a part of the needle hub 32 (for example, the needle hub body 38) has transparency such that the blood flowing into the lumen 32a of the needle hub 32 can be visually recognized from the outside of the needle hub 32. That is, the needle hub 32 is made of a transparent or translucent material.

As illustrated in FIG. 4, such a catheter assembly 10 further includes a restriction mechanism 54 that restricts relative axial movement of the catheter hub 18 and the needle hub 32 along the axial direction in the initial state of the catheter assembly 10. The restriction mechanism 54 is formed to release the restriction of the relative axial movement by rotating the needle hub 32 along the circumferential direction of the inner needle 30 with respect to the catheter hub 18 from the initial state of the catheter assembly 10.

In FIGS. 4 to 6, the restriction mechanism 54 includes a recess 56 formed on the inner surface of the catheter hub 18 and a protrusion 58 provided on the outer surface of the insertion portion 42 of the needle hub 32 and insertable into the recess 56. The recess 56 and the protrusion 58 restrict the relative axial movement of the catheter hub 18 and the needle hub 32 when the protrusion 58 comes into contact with a wall surface forming the recess 56.

The recess 56 is formed on the inner surface of the proximal cylindrical portion 26. The recess 56 includes a restriction groove 60 extending along the circumferential direction of the inner needle 30 and a release groove 62 extending from the restriction groove 60 in the proximal direction. The restriction groove 60 extends 180° or more in the circumferential direction of the inner needle 30 (see FIG. 5). The wall surface forming the restriction groove 60 includes two groove side surfaces 60a and 60b facing each other in the axial direction, a first end surface 60c located at one end in the extending direction of the restriction groove 60, and a second end surface 60d located at the other end in the extending direction of the restriction groove 60. The groove side surface 60a is located on the distal end side of the groove side surface 60b. The groove side surface 60a is smoothly connected to the stepped surface 28 without a step. The groove side surface 60a and the stepped surface 28 form one flat surface.

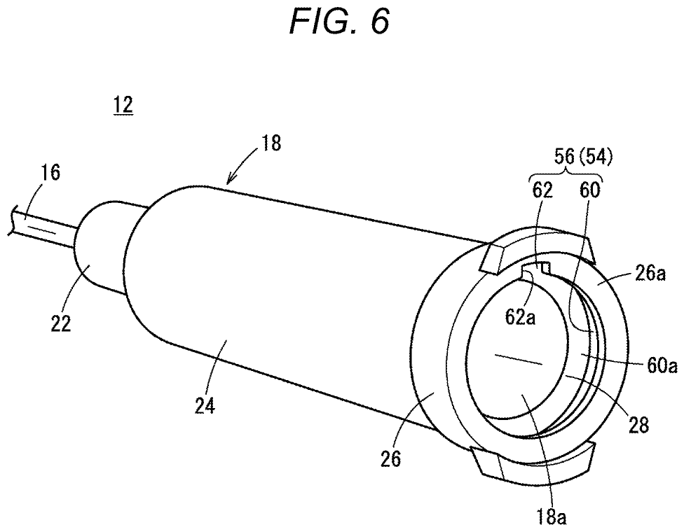

The release groove 62 extends linearly along the axial direction from one end portion of the restriction groove 60 in the extending direction to the proximal end surface 26a of the proximal cylindrical portion 26. That is, the release groove 62 is open to the proximal end surface 26a of the proximal cylindrical portion 26 (see FIGS. 4 and 6). The groove depth of the release groove 62 is the same as the groove depth of the restriction groove 60. The groove side surface 62a (see FIG. 6) forming the release groove 62 is smoothly connected to the first end surface 60c without a step. The groove side surface 62a and the first end surface 60c form one flat surface.

As illustrated in FIGS. 4, 5, and 7, the protrusion 58 protrudes radially outward from the distal end portion of the protruding end surface of one second protruding portion 50. In FIG. 5, the width dimension W1 of the protrusion 58 is the same as the width dimension of the second protruding portion 50. The width dimension W1 of the protrusion 58 is the same as or slightly smaller than the groove width W2 of the release groove 62 along the circumferential direction of the catheter hub 18. In FIG. 4, the length dimension L of the protrusion 58 along the axial direction of the inner needle 30 is substantially the same as the groove width W3 (the interval between the groove side surface 60a and the groove side surface 60b) of the restriction groove 60 along the axial direction of the catheter hub 18. The protruding length of the protrusion 58 from the protruding end surface of the second protruding portion 50 is substantially the same as the groove depth of each of the restriction groove 60 and the release groove 62. However, the protruding length of the protrusion 58 may be shorter than the groove depth of each of the restriction groove 60 and the release groove 62.

In the initial state of the catheter assembly 10, the protrusion 58 is located at the other end portion in the extending direction of the restriction groove 60 (an end portion opposite to the end portion with which the release groove 62 communicates). That is, in this state, the protrusion 58 is in contact with or in proximity to the second end surface 60d.

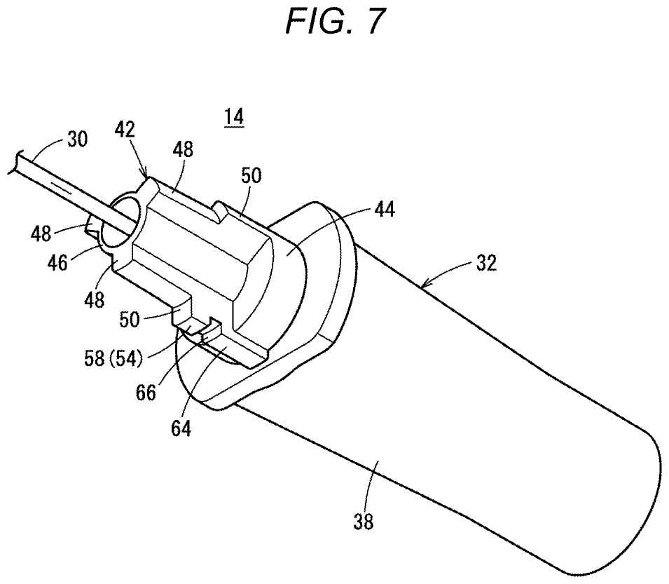

As illustrated in FIGS. 4 and 7, on the protruding end surface of the second protruding portion 50 provided with the protrusion 58, a proximal protrusion 64 protrudes radially outward with a space in the proximal direction from the protrusion 58. A recess-shaped notch portion 66 is formed between the protrusion 58 and the proximal protrusion 64. In the initial state of the catheter assembly 10, a portion 67 located on the proximal end side of the restriction groove 60 in the proximal cylindrical portion 26 is inserted into the notch portion 66 (see FIG. 4). The protruding length of the proximal protrusion 64 is the same as the protruding length of the protrusion 58.

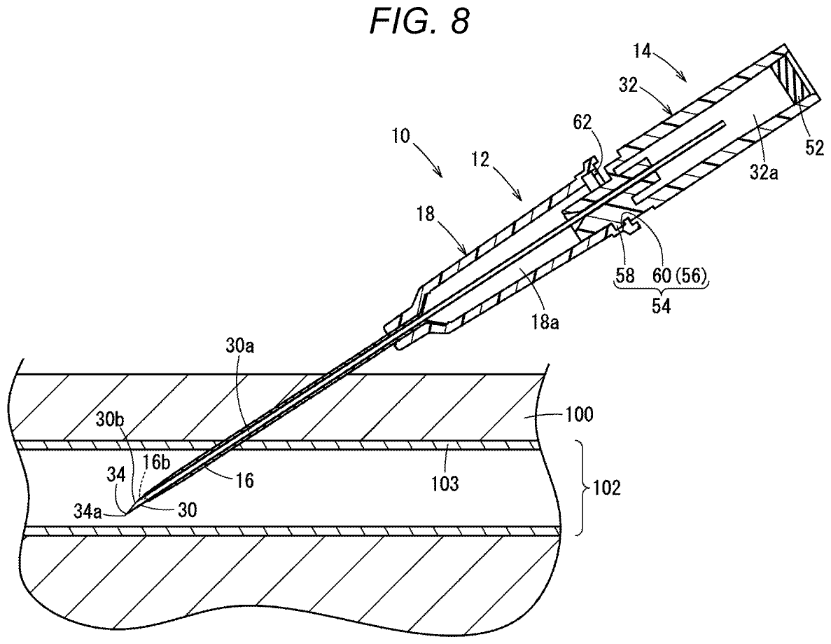

Next, a procedure using the catheter assembly 10 configured as described above will be described. As illustrated in FIG. 8, the user punctures a blood vessel 102 through a skin 100 while keeping the catheter assembly 10 in the initial state. In the initial state of the catheter assembly 10, the blade surface 34 protrudes in the distal direction from the distal end opening 16b of the catheter shaft 16 in a state of facing upward. That is, the blade edge 34a is located at the lowermost end of the inner needle 30 in the circumferential direction.

At this time, the protrusion 58 formed on the needle hub 32 is inserted into a recess 56 (restriction groove 60) formed in the catheter hub 18 (see FIGS. 4 and 5). Therefore, the relative axial movement of the needle hub 32 and the catheter hub 18 is restricted by the protrusion 58 coming into contact with a wall surface (groove side surfaces 60a, 60b) forming the recess 56. That is, the relative axial movement of the inner needle 30 and the catheter hub 18 in the axial direction is restricted. Therefore, at the time of the puncture operation, even when the user moves the catheter assembly 10 back and forth in order to probe the blood vessel 102, the blade edge 34a can be inhibited from piercing the catheter shaft 16.

When the blade edge 34a penetrates a front wall 103 of the blood vessel 102 and the distal end opening 30b of the inner needle 30 is located in the blood vessel 102, the blood in the blood vessel 102 flows back to the lumen 32a of the needle hub 32 via the lumen 30a of the inner needle 30. Then, the user can recognize that the blood vessel 102 is secured by the inner needle 30 by visually recognizing the backflow (flashback) of blood into the lumen 32a of the needle hub 32 from the outside of the needle hub 32.

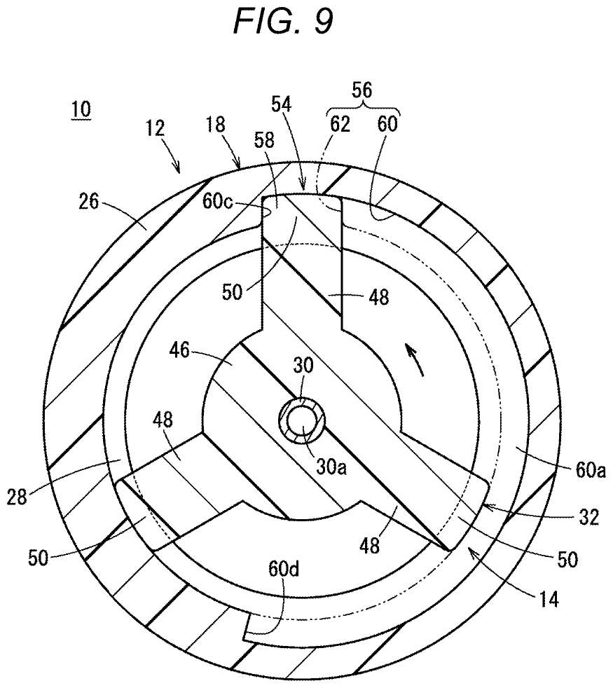

Thereafter, the user performs a removal operation. That is, the user rotates the needle hub 32 by a predetermined angle (180°) along the circumferential direction of the inner needle 30 with respect to the catheter hub 18 from the initial state of the catheter assembly 10. Then, as illustrated in FIG. 9, the protrusion 58 rotates by 180° from the other end portion to the one end portion of the restriction groove 60.

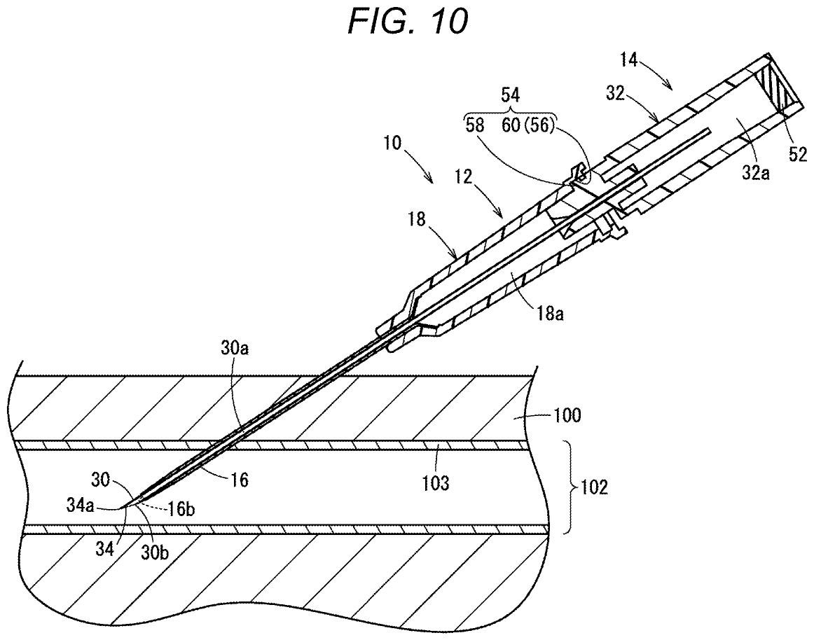

As a result, the protrusion 58 is located on the distal end side of the release groove 62, so that the restriction of the relative axial movement of the catheter hub 18 and the needle hub 32 is released. That is, the needle hub 32 is movable in the proximal direction with respect to the catheter hub 18. In addition, the inner needle 30 rotates by 180° around its axis. Therefore, as illustrated in FIG. 10, the blade surface 34 facing upward in the initial state of the catheter assembly 10 faces downward. That is, the blade edge 34a moves upward as compared with the initial state of the catheter assembly 10.

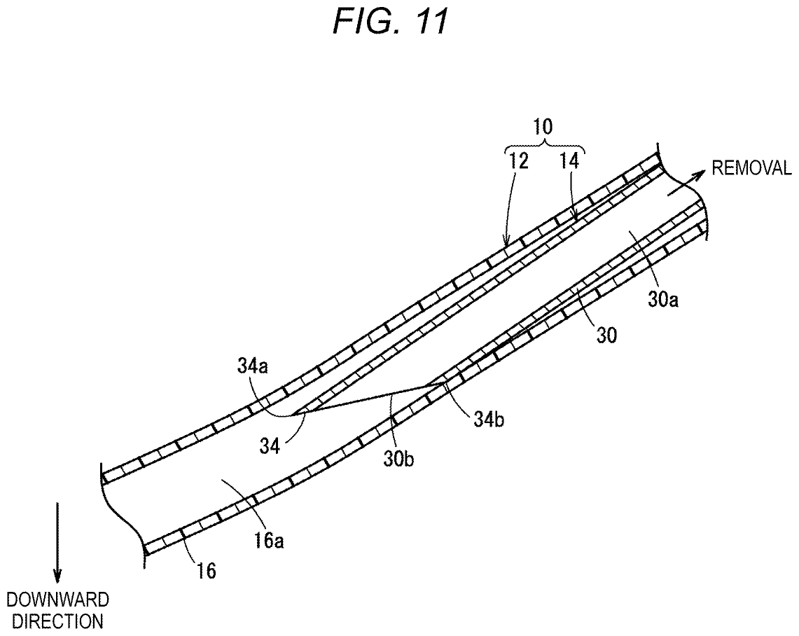

Subsequently, the user retracts the needle hub 32 with respect to the catheter hub 18. As a result, the protrusion 58 is separated from the recess 56 via the release groove 62, and the inner needle 30 is removed from the catheter shaft 16. At this time, the blade surface 34 faces downward. Therefore, as illustrated in FIG. 11, even when the catheter shaft 16 is curved downward, the blade edge 34a can be separated from the inner surface of the catheter shaft 16. Therefore, in the removal operation, the blade edge 34a is inhibited from piercing the catheter shaft 16.

In this case, the present embodiment has the following effects.

The catheter assembly 10 includes the restriction mechanism 54 that restricts the relative axial movement of the catheter hub 18 and the needle hub 32 along the axial direction in the initial state of the catheter assembly 10. The restriction mechanism 54 is formed so that the restriction of the relative axial movement is released by rotating the needle hub 32 with respect to the catheter hub 18 along the circumferential direction of the inner needle 30 from the initial state of the catheter assembly 10.

According to such a configuration, because the relative axial movement of the catheter hub 18 and the needle hub 32 is restricted by the restriction mechanism 54 in the initial state of the catheter assembly 10, the relative axial movement of the inner needle 30 and the catheter hub 18 is restricted in the puncture operation. Therefore, in the puncturing operation, it is possible to inhibit the blade edge 34a from piercing the catheter shaft 16.

After completion of the puncturing operation, the user rotates the needle hub 32 with respect to the catheter hub 18 along the circumferential direction of the inner needle 30 to release the restriction of the relative axial movement. As a result, because the inner needle 30 rotates around the axis, the direction of the blade surface 34 is changed. That is, the blade edge 34a moves upward. Therefore, even when the catheter shaft 16 is curved downward during the removal operation, the blade edge 34a can be separated from the inner surface of the catheter shaft 16. Therefore, in the removal operation, it is possible to inhibit the blade edge 34a from piercing the catheter shaft 16.

The restriction mechanism 54 is formed to release the restriction of the relative axial movement by rotating the needle hub 32 by 180° along the circumferential direction of the inner needle 30 with respect to the catheter hub 18 from the initial state of the catheter assembly 10.

According to such a configuration, the blade surface 34 can be directed downward by releasing the restriction of the relative axial movement of the catheter hub 18 and the needle hub 32. Therefore, in the removal operation, it is possible to effectively inhibit the blade edge 34a from piercing the catheter shaft 16.

The restriction mechanism 54 includes the recess 56 formed in the catheter hub 18 and the protrusion 58 formed in the needle hub 32 and inserted into the recess 56 in the initial state of the catheter assembly 10. The recess 56 and the protrusion 58 restrict relative axial movement when the protrusion 58 comes into contact with the wall surfaces (groove side surfaces 60a, 60b) forming the recess 56.

According to such a configuration, the configuration of the restriction mechanism 54 can be simplified.

The needle hub 32 has the insertion portion 42 to be inserted into the lumen 18a of the catheter hub 18 in the initial state of the catheter assembly 10. The recess 56 is formed on the inner surface of the catheter hub 18, and the protrusion 58 is formed on the outer surface of the insertion portion 42.

According to such a configuration, the restriction mechanism 54 can be formed compactly. In addition, because the protrusion 58 is not provided on the inner surface of the catheter hub 18, after the needle member 14 is removed from the catheter member 12, another medical device can be easily fitted into the lumen 18a of the catheter hub 18.

The recess 56 includes the restriction groove 60 extending along the circumferential direction of the inner needle 30, and the protrusion 58 moves along the restriction groove 60 as the needle hub 32 rotates with respect to the catheter hub 18.

According to such a configuration, the needle hub 32 can be smoothly rotated in the circumferential direction of the inner needle 30 along the restriction groove 60.

The recess 56 includes the release groove 62 extending from the restriction groove 60 to the proximal end surface of the catheter hub 18 (the proximal end surface 26a of the proximal cylindrical portion 26).

According to such a configuration, the protrusion 58 can be easily separated from the restriction groove 60 via the release groove 62.

The present invention is not limited to the above-described embodiments, and various modifications can be made without departing from the gist of the present invention.

The number, position, size, and shape of the recesses 56 and the protrusions 58 can be appropriately changed. In the present invention, the catheter assembly may include a restriction mechanism including a recess formed on the outer surface of the insertion portion of the needle hub and a protrusion formed on the inner surface of the catheter hub and inserted into the recess in the initial state of the catheter assembly.

The above-described embodiments are summarized as follows.

According to one embodiment, a catheter assembly (10) includes an inner needle (30), a needle hub (32) fixed to a proximal end portion of the inner needle, a tubular catheter shaft (16) through which the inner needle is inserted, and a hollow catheter hub (18) through which the inner needle is inserted and that is fixed to a proximal end portion of the catheter shaft, a blade surface (34) inclined with respect to an axis of the inner needle being formed at a distal end portion of the inner needle, the catheter assembly further including a restriction mechanism (54) that restricts relative axial movement of the catheter hub and the needle hub along an axial direction in an initial state of the catheter assembly, wherein the restriction mechanism is formed so that the restriction of the relative axial movement is released when the needle hub is rotated with respect to the catheter hub along a circumferential direction of the inner needle from the initial state of the catheter assembly.

In the above-described catheter assembly, the restriction mechanism may be formed such that the restriction of the relative axial movement is released by rotating the needle hub by 180° along the circumferential direction of the inner needle with respect to the catheter hub from the initial state of the catheter assembly.

In the above-described catheter assembly, the restriction mechanism may include a recess (56) formed in any one of the catheter hub and the needle hub, and a protrusion (58) formed in the other of the catheter hub and the needle hub and inserted into the recess in an initial state of the catheter assembly, and the recess and the protrusion may restrict the relative axial movement by the protrusion coming into contact with a wall surface forming the recess.

In the above-described catheter assembly, the needle hub may have an insertion portion (42) to be inserted into a lumen (18a) of the catheter hub in the initial state of the catheter assembly, one of the recess and the protrusion may be formed on an inner surface of the catheter hub, and the other of the recess and the protrusion may be formed on an outer surface of the insertion portion.

In the above-described catheter assembly, the recess may include a restriction groove (60) extending along a circumferential direction of the inner needle, and the protrusion may move along the restriction groove with rotation of the needle hub with respect to the catheter hub.

In the above-described catheter assembly, the recess may include a release groove (62) extending from the restriction groove to a proximal end surface (26a) of the catheter hub.

What is claimed is:

1. A catheter assembly comprising:
an inner needle comprising, at a distal end portion of the inner needle, a blade surface that is inclined with respect to an axis of the inner needle;
a needle hub fixed to a proximal end portion of the inner needle;
a tubular catheter shaft through which the inner needle is inserted;
a hollow catheter hub through which the inner needle is inserted and that is fixed to a proximal end portion of the catheter shaft; and
a restriction mechanism configured to restrict relative axial movement of the catheter hub and the needle hub along an axial direction in an initial state of the catheter assembly; wherein:
the restriction mechanism is configured such that the restriction of the relative axial movement is released when the needle hub is rotated with respect to the catheter hub along a circumferential direction of the inner needle from the initial state of the catheter assembly.

2. The catheter assembly according to claim 1, wherein:
the restriction mechanism is configured to release the restriction of the relative axial movement upon rotation of the needle hub by 180° along the circumferential direction of the inner needle with respect to the catheter hub from the initial state of the catheter assembly.

3. The catheter assembly according to claim 1, wherein:
the restriction mechanism comprises:
a recess located in one of the catheter hub or the needle hub, and
a protrusion located on the other of the catheter hub and the needle hub and inserted in the recess in an initial state of the catheter assembly; and
the recess and the protrusion restrict the relative axial movement by the protrusion contacting a wall surface defining the recess.

4. The catheter assembly according to claim 3, wherein:
the needle hub comprises an insertion portion located in a lumen of the catheter hub in an initial state of the catheter assembly;
one of the recess or the protrusion is located at an inner surface of the catheter hub; and
the other of the recess and the protrusion is located at an outer surface of the insertion portion.

5. The catheter assembly according to claim 4, wherein:
the recess comprises a restriction groove extending along a circumferential direction of the inner needle; and
the protrusion is configured to move along the restriction groove along with rotation of the needle hub with respect to the catheter hub.

6. The catheter assembly according to claim 5, wherein:
the recess comprises a release groove extending from the restriction groove to a proximal end surface of the catheter hub.

7. A catheter assembly comprising:
an inner needle comprising, at a distal end portion of the inner needle, a blade surface that is inclined with respect to an axis of the inner needle;
a needle hub fixed to a proximal end portion of the inner needle;
a tubular catheter shaft through which the inner needle is inserted; and
a hollow catheter hub through which the inner needle is inserted and that is fixed to a proximal end portion of the catheter shaft; wherein:
a recess is formed at an inner surface of the catheter hub; and
a protrusion is formed at an outer surface of the needle hub and inserted in the recess in an initial state of the catheter assembly;
the recess and the protrusion restrict relative axial movement of the catheter hub and the needle hub along an axial direction in an initial state of the catheter assembly by the protrusion contacting a wall surface defining the recess; and
the recess and the protrusion are configured such that the restriction of the relative axial movement is released when the needle hub is rotated with respect to the catheter hub along a circumferential direction of the inner needle from the initial state of the catheter assembly.

8. The catheter assembly according to claim 7, wherein:
the recess and the protrusion are configured to release the restriction of the relative axial movement upon rotation of the needle hub by 180° along the circumferential direction of the inner needle with respect to the catheter hub from the initial state of the catheter assembly.

9. The catheter assembly according to claim 7, wherein:
the needle hub comprises an insertion portion located in a lumen of the catheter hub in an initial state of the catheter assembly; and
the protrusion is located at an outer surface of the insertion portion.

10. The catheter assembly according to claim 9, wherein:
the recess comprises a restriction groove extending along a circumferential direction of the inner needle; and
the protrusion is configured to move along the restriction groove along with rotation of the needle hub with respect to the catheter hub.

11. The catheter assembly according to claim 10, wherein:
the recess comprises a release groove extending from the restriction groove to a proximal end surface of the catheter hub.

12. A method of using a catheter assembly, the method comprising:
providing the catheter assembly, which comprises:
an inner needle comprising, at a distal end portion of the inner needle, a blade surface that is inclined with respect to an axis of the inner needle,
a needle hub fixed to a proximal end portion of the inner needle,
a tubular catheter shaft through which the inner needle is inserted,
a hollow catheter hub through which the inner needle is inserted and that is fixed to a proximal end portion of the catheter shaft, and
a restriction mechanism configured to restrict relative axial movement of the catheter hub and the needle hub along an axial direction in an initial state of the catheter assembly;
puncturing a blood vessel of a patient while the catheter assembly is in the initial state, wherein, in the initial state, the blade surface protrudes in a distal direction from a distal end opening of the catheter shaft, and the blade surface faces upward;
rotating the needle hub along a circumferential direction of the inner needle from the initial state of the catheter assembly, such that the restriction of the relative axial movement is released, and such that the blade surface faces downward; and
retracting the needle hub with respect to the catheter hub such that the inner needle is removed from the catheter shaft.
